(19) United States
(12) Patent Application Publication
(10) Pub. No.: US 2022/0096978 A1
(43) Pub. Date: Mar. 31, 2022

(54) METHOD FOR PRODUCING AN ORGANIC SOLVENT

(71) Applicant: NISSAN CHEMICAL CORPORATION, Tokyo (JP)

(72) Inventors: Tomoya OHASHI, Toyama-shi (JP); Toyoshiro YOSHIDA, Nagoya-shi (JP); Suguru SASSA, Toyama-shi (JP)

(73) Assignee: NISSAN CHEMICAL CORPORATION, Tokyo (JP)

(21) Appl. No.: 17/429,989

(22) PCT Filed: Mar. 3, 2020
(86) PCT No.: PCT/JP2020/008972

§ 371 Date: Aug. 11, 2021

(30) Foreign Application Priority Data
Mar. 11, 2019 (JP) 2019-044162

(51) Int. Cl. B01D 39/16; B01D 29/11; C07C 41/36

(52) U.S. Cl. CPC B01D 39/163; B01D 29/11; B01D 2239/065; B01D 2239/0407; B01D 2239/0618; C07C 41/36

(57) ABSTRACT

A production method for an organic solvent that contains less metal impurities which when manufacturing a semiconductor device. This production method passes a liquid through a filter cartridge, wherein the filter cartridge is obtained by layering types of base cloths for filtration, wherein the base cloths for filtration are nonwoven fabric obtained by chemically bonding a metal adsorption group to polyolefin fibers, the base cloths for filtration include nonwoven fabric layer A and nonwoven fabric layer B, the nonwoven fabric layer A includes polyolefin fibers to which a sulfonate group is chemically bonded as a metal adsorption group, the nonwoven fabric layer B includes polyolefin fibers bonded thereto as a metal adsorption group at least one selected from the group consisting of an amino group, an N-methyl-D-glucamine group, an iminodiacetate group, an iminodiethanol group, an amidoxime group, a phosphate group, a carboxylate group, and an ethylene diamine triacetate group.

### TECHNICAL FIELD

The present invention relates to a method for producing an organic solvent having reduced metal impurities that would cause defects in the lithography step in the production of a semiconductor device.

### BACKGROUND ART

The organic solvent used in the lithography step in the production of a semiconductor device is required to be reduced in metal impurities that would cause very small defective portions (for example, called a defect having a size of about 1 to 100 nm) on a wafer. Patent Literature 1 discloses a filter having a high efficiency of adsorption and removal of metals.

### CITATION LIST

### Patent Literature

Patent Literature 1: JP 2018-167223 A

### SUMMARY OF INVENTION

### Technical Problem

There are provided a method for producing an organic solvent having reduced metal impurities that would cause defects on a wafer in the lithography step in the production of a semiconductor device, and a method for reducing metals in an organic solvent.

### Solution to Problem

The present invention embraces the followings.

[1] A method for producing an organic solvent, comprising the step of passing a solvent through a metal removing filter cartridge,

the metal removing filter cartridge being a filter cartridge having a plurality of types of filtration base fabric stacked or wound round a hollow cylinder,

wherein the filtration base fabric is nonwoven fabric having a metal adsorbing group chemically bonded to a polyolefin fiber, and

the filtration base fabric comprises a nonwoven fabric layer A and a nonwoven fabric layer B,

wherein the nonwoven fabric layer A comprises a polyolefin fiber having a sulfonic acid group chemically bonded as a metal adsorbing group, and

wherein the nonwoven fabric layer B comprises a polyolefin fiber having, as a metal adsorbing group, chemically bonded at least one member selected from the group consisting of an amino group, an N-methyl-D-glucamine group, an iminodiacetic acid group, an iminodiethanol group, an amidoxime group, a phosphoric acid group, a carboxylic acid group, and an ethylenediaminetriacetic acid group.

[2] The method for producing an organic solvent according to item [1] above, further comprising the step of passing a solvent through a fine-particle removing filter cartridge.

[3] The method for producing an organic solvent according to item [2] above, wherein a material for the fine-particle removing filter is at least one member selected from the group consisting of polyethylene and nylon.

[4] The method for producing an organic solvent according to any one of items [1] to [3] above, wherein the organic solvent is an organic solvent for use in forming a resist underlying film.

[5] The method for producing an organic solvent according to item [4] above, wherein the organic solvent is at least one member selected from the group consisting of propylene glycol monomethyl ether, propylene glycol monomethyl ether acetate, propylene glycol monoethyl ether, γ-butyrolactone, ethyl lactate, butyl lactate, and cyclohexanone.

[6] A method for reducing metals in an organic solvent to be purified, comprising passing an organic solvent to be purified through a metal removing cartridge filter to reduce metals in the organic solvent,

the filter cartridge having a plurality of types of filtration base fabric stacked or wound round a hollow cylinder,

wherein the filtration base fabric is nonwoven fabric having a metal adsorbing group chemically bonded to a polyolefin fiber, and

the filtration base fabric comprises a nonwoven fabric layer A and a nonwoven fabric layer B,

wherein the nonwoven fabric layer A comprises a polyolefin fiber having a sulfonic acid group chemically bonded as a metal adsorbing group, and

wherein the nonwoven fabric layer B comprises a polyolefin fiber having, as a metal adsorbing group, chemically bonded at least one member selected from the group consisting of an amino group, an N-methyl-D-glucamine group, an iminodiacetic acid group, an iminodiethanol group, an amidoxime group, a phosphoric acid group, a carboxylic acid group, and an ethylenediaminetriacetic acid group.

### Advantageous Effects of Invention

By performing the production of an organic solvent using the filter cartridge described in the present invention, an organic solvent having markedly reduced metal impurities can be produced. By using the produced organic solvent, it is possible to reduce various defects caused in the lithography step in the semiconductor production process.

### DESCRIPTION OF EMBODIMENTS

<Method for Producing an Organic Solvent>

The method for producing an organic solvent of the present invention comprises the step of passing an organic solvent to be purified, which is in a solution state at room temperature, through the metal removing filter cartridge described below in detail.

The step of passing the organic solvent to be purified may be performed by, for example, passing the organic solvent to be purified, which is commercially available, through a metal removing filter cartridge directly connected (at two portions, i.e., an inlet and an outlet) to the production apparatus (vessel for production) in which the organic solvent is used. The step of passing the organic solvent to be purified may be performed once, twice or more times. The step of passing the organic solvent to be purified is preferably circulation filtration using a pump. It is preferred that the organic solvent is passed through both the metal removing filter cartridge in the present invention and a fine-particle removing filter cartridge and circulated, wherein the fine-particle removing filter cartridge is connected in series to the metal removing filter cartridge. The time required for the circulation is, for example, within the range of 3 to 144 hours. The filtration flow rate is, for example, within the range of 1 to 1,000 L/hour.

<Organic Solvent to be Purified>

With respect to the organic solvent to be purified in the present invention, it is recommended that the organic solvent used be, for example, an organic solvent generally used in the below-mentioned lithography step; however, the organic solvent to be purified is not limited to these solvents.

Examples of the organic solvents to be purified include ethylene glycol monomethyl ether, ethylene glycol monoethyl ether, methyl cellosolve acetate, ethyl cellosolve acetate, diethylene glycol monomethyl ether, diethylene glycol monoethyl ether, propylene glycol, propylene glycol monomethyl ether, propylene glycol monoethyl ether, propylene glycol monomethyl ether acetate, propylene glycol propyl ether acetate, toluene, xylene, methyl ethyl ketone, methyl isobutyl ketone, cyclopentanone, cyclohexanone, cycloheptanone, 4-methyl-2-pentanol, methyl 2-hydroxyisobutyrate, ethyl 2-hydroxyisobutyrate, ethyl ethoxyacetate, 2-hydroxyethyl acetate, methyl 3-methoxypropionate, ethyl 3-methoxypropionate, ethyl 3-ethoxypropionate, methyl 3-ethoxypropionate, methyl pyruvate, ethyl pyruvate, ethyl acetate, butyl acetate, ethyl lactate, butyl lactate, 2-heptanone, methoxycyclopentane, anisole, γ-butyrolactone, N-methylpyrrolidone, N,N-dimethylformamide, and N,N-dimethylacetamide. These solvents may be used each alone or in combination of two or more.

Of these solvents, preferred are propylene glycol monomethyl ether, propylene glycol monomethyl ether acetate, propylene glycol monoethyl ether, γ-butyrolactone, ethyl lactate, butyl lactate, and cyclohexanone. Especially preferred are propylene glycol monomethyl ether, propylene glycol monomethyl ether acetate, and propylene glycol monoethyl ether.

<Filter Cartridge>

The filter cartridge in the present invention is preferably one which is described in JP 2018-167223 A.

The filter cartridge in the present invention is a filter cartridge having a plurality of types of filtration base fabric stacked or wound round a hollow cylinder, wherein the filtration base fabric is nonwoven fabric having a metal adsorbing group chemically bonded to a polyolefin fiber, and the filtration base fabric comprises a nonwoven fabric layer A and a nonwoven fabric layer B, wherein the nonwoven fabric layer A comprises a polyolefin fiber having a sulfonic acid group chemically bonded as a metal adsorbing group, and wherein the nonwoven fabric layer B comprises a polyolefin fiber having, as a metal adsorbing group, chemically bonded at least one member selected from the group consisting of an amino group, an N-methyl-D-glucamine group, an iminodiacetic acid group, an iminodiethanol group, an amidoxime group, a phosphoric acid group, a carboxylic acid group, and an ethylenediaminetriacetic acid group.

The filter cartridge in the present invention is a filter cartridge having a plurality of types of filtration base fabric stacked or wound round a hollow cylinder, wherein the filtration base fabric is nonwoven fabric having a metal adsorbing group chemically bonded to a polyolefin fiber, and the filtration base fabric comprises nonwoven fabric layer A and nonwoven fabric layer B. Nonwoven fabric layer A comprises a polyolefin fiber having a sulfonic group chemically bonded as a metal adsorbing group, and nonwoven fabric layer B comprises a polyolefin fiber having, as a metal adsorbing group, chemically bonded at least one member selected from the group consisting of an amino group, an N-methyl-D-glucamine group, an iminodiacetic acid group, an iminodiethanol group, an amidoxime group, a phosphoric acid group, a carboxylic acid group, and an ethylenediaminetriacetic acid group. By virtue of this, metals can be efficiently removed. A plurality of types of filtration base fabric includes a single piece of filtration base fabric integrated by binding together different types of filtration base fabric.

In the present invention, nonwoven fabric layer B especially preferably comprises a polyolefin fiber having an iminodiethanol group chemically bonded thereto. This is because such a polyolefin fiber has high efficiency of removal of metals. With respect to the metals that the group can adsorb, the sulfonic acid group adsorbs mainly Na, Cu, and K, and the iminodiethanol group adsorbs mainly Cr, Al, and Fe.

The polyolefin fiber constituting nonwoven fabric layers A and B is preferably a continuous fiber. This is because continuous fiber nonwoven fabric is unlikely to cause fabric tailings and has high filter performance. Especially, preferred is melt-blown continuous fiber nonwoven fabric having a weight per unit area (basis weight) of 10 to 100 g/m².

The polyolefin fiber constituting nonwoven fabric layers A and B preferably has a single fiber average diameter of 0.2 to 10 μm. When the single fiber average diameter of the polyolefin fiber is in the above range, high filter performance is expected. In addition, the surface area (specific surface area) of the fiber can be increased, so that the surface of the substrate for a graft polymerization reaction is increased, and thus an increase of the graft ratio can be expected.

With respect to the polyolefin fiber, preferred is at least one member selected from the group consisting of polypropylene, a copolymer of propylene and ethylene, polyethylene, and a copolymer of ethylene and another α-olefin having 4 or more carbon atoms, and especially preferred is high density polyethylene. These polymers are inert and stable with respect to a chemical liquid, and capable of undergoing graft polymerization.

It is preferred that the filter cartridge is a filter cartridge comprising a hollow cylinder and filtration base fabric, wherein the filtration base fabric is nonwoven fabric having a metal adsorbing group chemically bonded to a polyolefin fiber, and wherein the filtration base fabric is wound round the hollow cylinder to form a stacked structure.

The filter in the present invention is a filter having the above-mentioned filter cartridge incorporated. For example, the filter cartridge has filtration base fabric wound round a cylinder and is contained in a container. When incorporating the filter cartridge into a container for filter, for example, the filter cartridge may be contained in the container, which may then be incorporated into the filter. In the case of a cartridge type filter, the filter function can be regenerated by replacing only the filter cartridge. The present invention includes, for example, a capsule type filter such that the container for filter including the contents is replaced. In the case of a capsule type filter, a filtration portion corresponds to the filter cartridge.

The method for causing a functional group to be chemically bonded to a polyolefin fiber is described below. Examples of the methods include a method in which a polyolefin fiber is irradiated with an electron beam or radiation, such as a γ-ray, and then contacted with an emulsion containing a reactive monomer, such as GMA; and a method in which a polyolefin fiber is contacted with an emulsion containing a reactive monomer, and then irradiated with an electron beam or radiation, such as a γ-ray, causing graft polymerization of the reactive monomer on the polyolefin fiber. When the polyolefin fiber is irradiated with an electron beam, the irradiation dose achieved may be within the range of generally 1 to 200 kGy, preferably 5 to 100 kGy, more preferably 10 to 50 kGy. The irradiation is preferably conducted in a nitrogen gas atmosphere. As an electron beam irradiation apparatus, one which is commercially available may be used; and, for example, as an area beam-type electron beam irradiation apparatus, EC250/15/180L (manufactured by Iwasaki Electric Co., Ltd.), EC300/165/800 (manufactured by Iwasaki Electric Co., Ltd.), or EPS300 (manufactured by NHV Corporation) may be used.

Specific examples of the graft polymerization method include a liquid-phase graft polymerization method. In the liquid-phase graft polymerization method, nonwoven fabric is activated by irradiation with radiation, such as a γ-ray, or an electron beam; and then immersed in an emulsion containing water, a surfactant, and a reactive monomer, completing graft polymerization on the nonwoven fabric substrate. Subsequently, a functional group, such as a sulfonic acid group, an amino group, an N-methyl-D-glucamine group, an iminodiacetic acid group, an iminodiethanol group, an amidoxime group, a phosphoric acid group, a carboxylic acid group, or an ethylenediaminetriacetic acid group, that is, an ion-exchange group and/or a chelate group is introduced into the graft chains formed on the substrate. In the present invention, the graft polymerization method is not particularly limited to a liquid-phase graft polymerization method; and there may be used, for example, a gas-phase graft polymerization method in which a substrate is contacted with vapor of a monomer to cause polymerization; or an impregnation gas-phase graft polymerization method in which a substrate is immersed in a monomer solution, and then removed from the monomer solution to cause a reaction in a gas phase. As chemical formulae of representative functional groups, a sulfonic acid group (SC group) is shown in (Chemical formula 1), an iminodiethanol group (IDE group) is shown in (Chemical formula 2), an iminodiacetic acid group (IDA group) is shown in (Chemical formula 3), and an N-methyl-D-glucamine group (NMDG group) is shown in (Chemical formula 4).

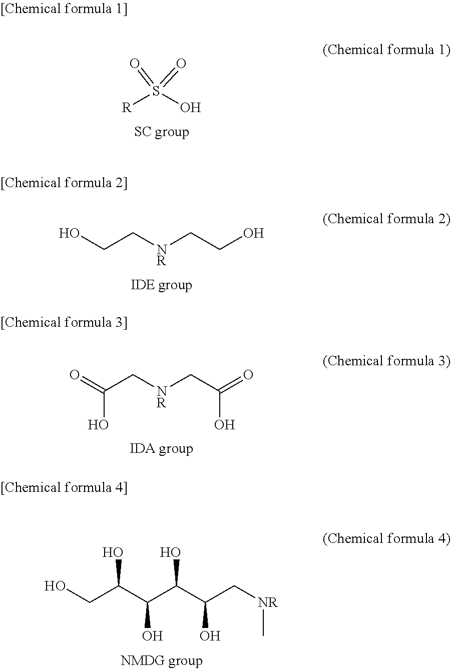

In the (Chemical formula 1) to (Chemical formula 3), R is the below-shown polyethylene (PE)+GMA (Chemical formula 5) or polypropylene (PP)+GMA (Chemical formula 6). R in the (Chemical formula 4) is a methyl group.

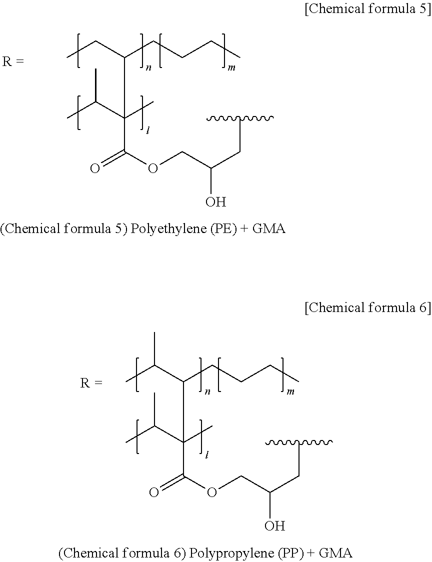

In the (Chemical formula 5) and (Chemical formula 6) above, n and m are an integer of 1 or more.

<Fine-Particle Removing Filter>

In the method for producing an organic solvent of the present invention, it is preferred that the organic solvent to be purified is passed through the filter cartridge, and then further passed through a fine-particle removing filter. With respect to the fine-particle removing filter, one which has been known may be used. The material for the fine-particle removing filter is preferably at least one member selected from the group consisting of polyethylene and nylon.

The fine-particle removing filter generally has a pore diameter of 30 nm or less, preferably, for example, 0.1 to 30 nm, for example, 0.1 to 20 nm, or, for example, 1 to 10 nm.

<Method for Reducing Metals>

The method of the present invention for reducing metals in an organic solvent to be purified comprises subjecting the above-mentioned organic solvent to be purified to filtration using a filter cartridge to reduce metals in the organic solvent,

the cartridge filter having a plurality of types of filtration base fabric stacked or wound round a hollow cylinder,

wherein the filtration base fabric is nonwoven fabric having a metal adsorbing group chemically bonded to a polyolefin fiber, and

the filtration base fabric comprises a nonwoven fabric layer A and a nonwoven fabric layer B,

wherein the nonwoven fabric layer A comprises a polyolefin fiber having a sulfonic acid group chemically bonded as a metal adsorbing group, and

wherein the nonwoven fabric layer B comprises a polyolefin fiber having, as a metal adsorbing group, chemically bonded at least one member selected from the group consisting of an amino group, an N-methyl-D-glucamine group, an iminodiacetic acid group, an iminodiethanol group, an amidoxime group, a phosphoric acid group, a carboxylic acid group, and an ethylenediaminetriacetic acid group.

By virtue of the step in the above method, the organic solvent to be purified can be reduced in metal impurities contained therein, which are derived from the raw materials or solvent, making it possible to reduce defects caused in the lithography step.

By the above-mentioned method for reducing metals, various metal impurities (for example, Na, Cu, Cr, Al, and Fe) can be reduced to, for example, 0.5 ppb or less, for example, 0.4 ppb or less.

The amount of the metal impurities contained can be determined by, for example, the method described in the Examples.

### EXAMPLES

Hereinbelow, the present invention will be described in more detail with reference to the following Examples and others, which should not be construed as limiting the scope of the present invention.

### Example 1

20 L of propylene glycol monomethyl ether (PM-P, manufactured by KH Neochem Co., Ltd.) as an organic solvent to be purified was subjected to filtration using one cartridge filter (10 inches) (manufactured by Kurashiki Textile Manufacturing Co., Ltd.) described in JP 2018-167223 A at a flow rate of 3 L per minute for 100 minutes. The metal content in the organic solvent obtained after the filtration was determined by means of an ICP-MS (Agilent 8800, manufactured by Agilent Technologies, Inc.).

### Comparative Example 1

The metal content in the organic solvent to be purified used in Example 1 without filtration was determined by the same procedures as in Example 1.

### Comparative Example 2

Filtration was conducted by substantially the same procedures as in Example 1 except that the cartridge filter used in Example 1 was replaced by a cartridge filter (nylon filter ABD1ANM3EH1 (20 nm nylon filter), manufactured by Nihon Pall Ltd.), and the metal content was determined by the same procedures as in Example 1.

### Comparative Example 3

Filtration was conducted by substantially the same procedures as in Example 1 except that the cartridge filter used in Example 1 was replaced by 20 kg of a strongly acidic ion-exchange resin (XSC-1115-H, manufactured by Muromachi Chemicals Inc.) and ion exchange was performed for 4 hours. Then, the metal content was determined by the same procedures as in Example 1.

<Metal Concentration of Organic Solvent>

The results of the measurement of the metal concentration after conducting the treatment method in Example 1 are shown in Table 1.

TABLE 1
Results of metal concentration measurement (ppb)

| | |
|---|---|
| Example 1 | 0.1 |
| Comparative Example 1 | 0.4 |
| Comparative Example 2 | 0.2 |
| Comparative Example 3 | 1.3 |

The results seen in Table 1 show that Example 1 can effectively reduce the metal concentration.

### Example 2

20 L of propylene glycol monomethyl ether acetate (EL-PGMEA, manufactured by Toyo Gosei Co., Ltd.) as an organic solvent to be purified was subjected to filtration using one cartridge filter (10 inches) (manufactured by Kurashiki Textile Manufacturing Co., Ltd.) described in JP 2018-167223 A at a flow rate of 2 L per minute for 50 minutes. The metal content of the solution obtained after the filtration was determined by means of an ICP-MS (Agilent 8800, manufactured by Agilent Technologies, Inc.).

### Comparative Example 4

The metal content of the solvent used in Example 1 without filtration was determined by the same procedures as in Example 1.

TABLE 2
Results of metal concentration measurement (ppb)

| | |
|---|---|
| Example 2 | 0.0 |
| Comparative Example 4 | 3.9 |

The results seen in Table 2 show that Example 2 can effectively reduce the metal concentration.

### INDUSTRIAL APPLICABILITY

By the present invention, there can be provided an organic solvent which is particularly reduced in the amount of metal impurities.

1. A method for producing an organic solvent, comprising the step of passing a solvent through a metal removing filter cartridge,
the metal removing filter cartridge being a filter cartridge having a plurality of types of filtration base fabric stacked or wound round a hollow cylinder,
wherein the filtration base fabric is nonwoven fabric having a metal adsorbing group chemically bonded to a polyolefin fiber, and
the filtration base fabric comprises a nonwoven fabric layer A and a nonwoven fabric layer B,
wherein the nonwoven fabric layer A comprises a polyolefin fiber having a sulfonic acid group chemically bonded as a metal adsorbing group, and
wherein the nonwoven fabric layer B comprises a polyolefin fiber having, as a metal adsorbing group, chemically bonded at least one member selected from the group consisting of an amino group, an N-methyl-D-glucamine group, an iminodiacetic acid group, an iminodiethanol group, an amidoxime group, a phosphoric acid group, a carboxylic acid group, and an ethylenediaminetriacetic acid group.

2. The method for producing an organic solvent according to claim 1, further comprising the step of passing a solvent through a fine-particle removing filter cartridge.

3. The method for producing an organic solvent according to claim 2, wherein a material for the fine-particle removing filter is at least one member selected from the group consisting of polyethylene and nylon.

4. The method for producing an organic solvent according to claim 1, wherein the organic solvent is an organic solvent for use in forming a resist underlying film.

5. The method for producing an organic solvent according to claim 4, wherein the organic solvent is at least one member selected from the group consisting of propylene glycol monomethyl ether, propylene glycol monomethyl ether acetate, propylene glycol monoethyl ether, γ-butyrolactone, ethyl lactate, butyl lactate, and cyclohexanone.

6. A method for reducing metals in an organic solvent to be purified, comprising passing an organic solvent to be purified through a metal removing cartridge filter to reduce metals in the organic solvent,
the filter cartridge having a plurality of types of filtration base fabric stacked or wound round a hollow cylinder,
wherein the filtration base fabric is nonwoven fabric having a metal adsorbing group chemically bonded to a polyolefin fiber, and
the filtration base fabric comprises a nonwoven fabric layer A and a nonwoven fabric layer B,
wherein the nonwoven fabric layer A comprises a polyolefin fiber having a sulfonic acid group chemically bonded as a metal adsorbing group, and
wherein the nonwoven fabric layer B comprises a polyolefin fiber having, as a metal adsorbing group, chemically bonded at least one member selected from the group consisting of an amino group, an N-methyl-D-glucamine group, an iminodiacetic acid group, an iminodiethanol group, an amidoxime group, a phosphoric acid group, a carboxylic acid group, and an ethylenediaminetriacetic acid group.
